(19) United States
(12) Patent Application Publication
(10) Pub. No.: US 2012/0244079 A1
(43) Pub. Date: Sep. 27, 2012

(54) TRACEABLE RETINOID ACID FOR IMAGING, DISEASE PREVENTION AND THERAPY

(75) Inventors: Shi Ke, Houston, TX (US); Michel Mawad, Houston, TX (US); Wei Wang, Houston, TX (US)

(73) Assignee: BAYLOR COLLEGE OF MEDICINE, Houston, TX (US)

(21) Appl. No.: 13/319,012

(22) PCT Filed: May 6, 2010
(86) PCT No.: PCT/US10/33918

§ 371 Date: Jun. 15, 2012

Related U.S. Application Data
(60) Provisional application No. 61/176,419, filed on May 7, 2009.

(51) Int. Cl. C07D 403/08; A61K 49/04; A61K 49/00

(52) U.S. Cl. 424/9.44; 548/455; 424/9.6; 424/9.1

(57) ABSTRACT

The present invention provides nonradioactive NIR optical imaging agents based up on the structure of retinoid acid derivatives. The nonradioactive NIR optical imaging agent has been evaluated at the cellular level by confocal microscopy, and in vivo in a whole animal using a various xenograft models. The specific uptake of this agent in human cancer cells and multiple xenograft models demonstrate that nonradioactive near infrared dye labeled retinoid metabolites and/or analogs are useful for early stage cancer studies and diagnoses. Also, the present invention provides that nonradioactive near infrared dye labeled retinoid metabolites and/or analogs are useful for visualization of drug redistribution within the body which is useful in determining the optimal biological dose. Ultimately, the visualization data can be used as an analytical tool to reduce any systemic toxicity.

### CROSS-REFERENCE TO RELATED APPLICATIONS

This application is a National Stage of PCT/US2010/033918 filed on May 6, 2010, which claims the benefit of U.S. Provisional Application Ser. No. 61/176,419, filed May 7, 2009, the entire contents of both are hereby incorporated herein by reference.

### STATEMENT REGARDING FEDERALLY SPONSORED RESEARCH OR DEVELOPMENT

This invention was made with government support under W81XWH-08-1-0489 awarded by the United States Department of Defense. The government has certain rights in the invention.

### TECHNICAL FIELD

The present disclosure relates generally to imaging agents and, in particular, non-radioactive imaging agents used to image and/or detect multiple human cancer xenografts. More specifically, the present disclosure concerns the imaging of human cancer cells and multiple xenograft models using a nonradioactive near infrared dye labeled retinoid metabolite and/or analog.

### BACKGROUND OF THE INVENTION

Cancer is a major public health problem in the United States (U.S.). Currently, one in 4 deaths in the U.S. is due to cancer. Oral, head and neck, esophageal, lung, colon, bladder cancers, and leukemia account for 35% of one and half million new cancer cases in 2008 alone. There is a need for an imaging agent that can be used in early diagnosis of all or any one of these cancers. An analysis of lung cancer can be used to illustrate the need and utility of such an imaging agent.

For example in the U.S., an estimated 215,000 new cases of lung cancer were diagnosed in 2008, and more than 161,000 lung cancer related deaths were reported. Lung cancer is the leading cause of cancer deaths for both men and women, causing more than colon, breast, and prostate cancers combined. Progress has been slow in lowering the high mortality of this disease due to difficulty in detection, and current therapies including surgery, radiation, and chemotherapy are not curative.

Early diagnosis of lung cancer is rare due to the absence of symptoms at this stage. Studies show that despite annual computed tomography (CT) screening, most individuals who died from lung cancer did not have their cancers detected early enough for initiating successful treatment. A screening test that would detect lung cancer in early stages could significantly reduce the morbidity and mortality of this disease. Therefore, the high incidence and mortality of lung cancer argue powerfully for the development of target-specific molecular imaging agents for detecting and treating lung cancers.

An attractive target-specific class of agents are retinoids. Retinoids are the most extensively studied chemopreventative agents due to their functions in regulating a range of physiological processes, especially hematopoiesis, cell growth and differentiation, proliferation, and apoptosis. Retinoids have been effectively used to treat oral cancers, head and neck, esophageal, lung, colon, and bladder cancers, leukemia, as well as in leukemia prevention. In general, the term retinoid refers to compounds of vitamin A metabolites and analogs, both synthetic and naturally occurring.

For example, retinoic acid can reverse oral premalignancy, significantly decrease second primary tumors, and provide a treatment benefit against smoking-related second primary tumors in the head, neck, lung, esophagus, and bladder. This benefit may last up to 3 years after the completion of treatment but disappears with longer follow-up. These data demonstrate the persistence of retinoic acid preventative effects and also suggest that cancer cells can uptake retinoids.

Although retinoid analogs have been modified for SPECT imaging, the particular modification presents a number of disadvantages not addressed in the prior art. Therefore, this disclosure present retinoids in a new light as potential non-radioactive tumor-imaging agents.

Optical imaging is an active and promising area for both in vitro and in vivo molecular imaging studies. Of the various optical imaging techniques used to date, near infrared (NIR) fluorescence imaging is particularly promising. The wavelength for near infrared light ranges from 700 to 900 nanometers with minimal autofluorescence, and is minimally absorbed by hemoglobin (the principal absorber of visible light), water, and lipids (the principal absorbers of infrared light). Considering the advantages of NIR imaging, this method could provide an attractive approach for improving the imaging accuracy and safety for patients.

The object of this disclosure is to provide nonradioactive NIR optical imaging agents based upon the structure of retinoid acid derivatives. The nonradioactive NIR optical imaging agent has been evaluated at the cellular level by confocal microscopy, and in vivo in a whole animal using a various xenograft models. The specific uptake of this agent in human cancer cells and multiple xenograft models demonstrate that nonradioactive near infrared dye labeled retinoid metabolites and/or analogs are useful for early stage cancer studies and diagnoses. Also, the nonradioactive near infrared dye labeled retinoid metabolites and/or analogs are useful for visualization of drug redistribution within the body which is useful in determining the optimal biological dose. Ultimately, the visualization data can be used as an analytical tool to reduce any systemic toxicity.

### BRIEF SUMMARY OF THE INVENTION

The present invention is directed to a composition and method which includes a retinoid analog, a spacer moiety, and a dye.

A general embodiment of the invention is a composition comprising a retinoid analog, a spacer moiety comprising a reactive amino functionality wherein the spacer moiety is chemically bonded to the retinoid analog and, a dye that is chemically bonded to the spacer moiety. In an embodiment of the invention, the chemical bond connecting the retinoid analog and the spacer moiety forms a ester, ether, thioether, thioester, amide, a carbon-carbon single bond, a carbon-carbon double bond, or a carbon-carbon triple bond. In further embodiment of the invention, the spacer moiety further comprises an alkyl_{(C=1-8)}, alkenyl_{(C=1-8)}, or aralkyl_{(C=1-8)}, or a substituted version of any of these groups. In an embodiment of the invention, the dye is a contrast agent and/or a fluorophore. In specific embodiments of the invention, the dye is IRdye800CW, IRdye800RS, IRdye700DX and/or a cyanine dye. In an embodiment of the invention, the retinoid analog has the formula:

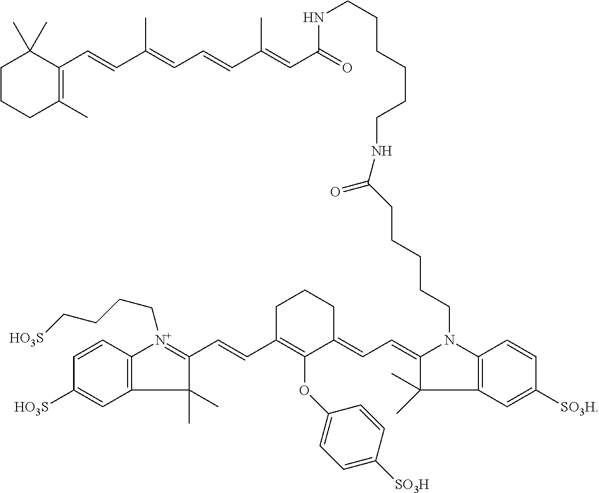

A general embodiment of the invention is a composition having the general formula:

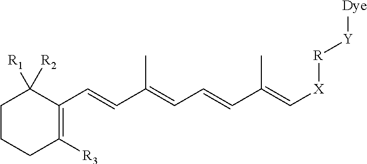

wherein R₁, R₂ and R₃ may be independently selected from alkyl_{(C=1-8)}, aryl_{(C=6-18)}, aralkyl_{(C=7-15)}, alkyl halide_{(C=1-8)}, alkenyl_{(C=1-8)}, substituted alkyl_{(C=1-8)}, substituted aryl_{(C=6-18)}, substituted aralkyl_{(C=6-18)}, substituted alkyl halide_{(C=1-8)}, substituted alkenyl_{(C=2-10)} or any combinations thereof; X is —NHCO—, —NH—, —OCO—, —O—, —S—, —CH₂CH₂—, —CH═CH—, or —C≡C—; R is alkyl_{(C=1-8)}, alkenyl_{(C=1-8)}, or aralkyl_{(C=1-8)}, a substituted version of any of these groups, or a substituted and functionalized version of any of these groups; Y is —NHCO— or —SCO—. In an embodiment of the invention, the dye is a contrast agent and/or a fluorophore. In specific embodiments of the invention, the dye is IRdye800CW, IRdye800RS, IRdye700DX and/or a cyanine dye. In a specific embodiment of the invention, R is further defined by the formula R₅-R₄—W, wherein W is —NH₂, a halogen, a hydroxy group, thiol and wherein W is coupled to X an amide, amine, ester, ether, thioether, ketone, a carbon-carbon single bond, a carbon-carbon double bond, or a carbon-carbon triple bond is formed; R₄ is alkyl_{(C=1-8)}, alkenyl_{(C=1-8)}, or aralkyl_{(C=1-8)}; and R₅ is —NH₂ or —SH and when coupled to the dye forms an amide bond or a thioester bond. X and W may also form an amide bond. In specific embodiments of the invention, R₄ is alkyl_{(C=5-8)}, Y is —NHCO—, and/or the compound is non-radioactive. The composition also may have the formula:

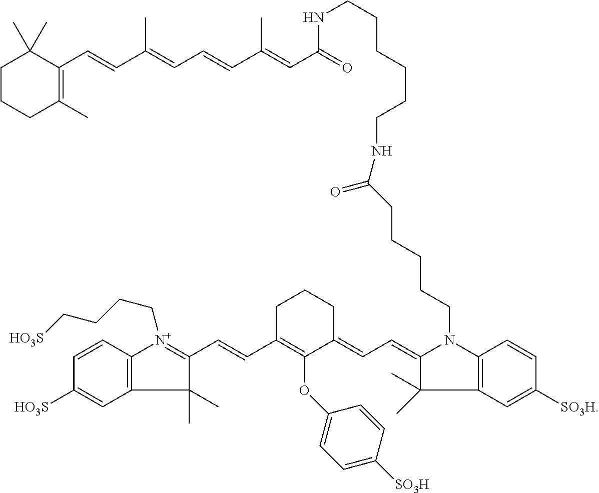

Another general embodiment of the invention is a method for near infrared imaging comprising the steps of: treating a subject with a compound having the general formula:

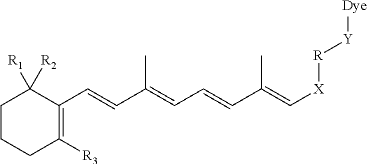

wherein R₁, R₂ and R₃ may be independently selected from alkyl_{(C=1-8)}, aryl_{(C=6-18)}, aralkyl_{(C=7-15)}, alkyl halide_{(C=1-8)}, alkenyl_{(C=1-8)}, substituted alkyl_{(C=1-8)}, substituted aryl_{(C=6-18)}, substituted aralkyl_{(C=6-18)}, substituted alkyl halide_{(C=1-8)}, substituted alkenyl_{(C=2-10)} or any combinations thereof; X is —NHCO—, —NH—, —OCO—, —O—, —S—, —CH₂CH₂—, —CH═CH—, or —C≡C—; R is alkyl_{(C=1-8)}, alkenyl_{(C=1-8)}, or aralkyl_{(C=1-8)}, a substituted version of any of these groups, or a substituted and functionalized version of any of these groups; and Y is —NHCO— or —SCO—. In an embodiment of the invention, the dye is a contrast agent and/or a fluorophore. In specific embodiments of the invention, the dye is IRdye800CW, IRdye800RS, IRdye700DX and/or a cyanine dye. In embodiments of the invention the compound is non-radioactive and/or the compound has the formula:

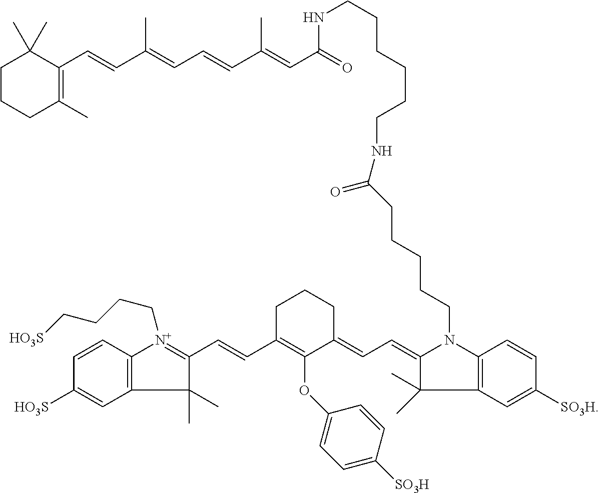

An embodiment of the invention is a method of diagnosing a disease in a patient comprising administering to the patient a composition comprising a retinoid analog, a spacer moiety comprising a reactive amino functionality wherein the spacer moiety is chemically bonded to the retinoid analog and, a dye that is chemically bonded to the spacer moiety, and detecting the location of composition in the patient. In an embodiment of the invention, the chemical bond connecting the retinoid analog and the spacer moiety forms a ester, ether, thioether, thioester, amide, a carbon-carbon single bond, a carbon-carbon double bond, or a carbon-carbon triple bond. In further embodiment of the invention, the spacer moiety further comprises an alkyl_{(C=1-8)}, alkenyl_{(C=1-8)}, or aralkyl_{(C=1-8)}, or a substituted version of any of these groups. In an embodiment of the invention, the dye is a contrast agent and/or a fluorophore. In an embodiment of the invention, the disease is cancer. In a specific embodiment of the invention, the disease is a retinoid receptor-positive disease. In an embodiment of the invention, the disease is cancer. In specific embodiments of the invention, the disease is colon cancer, lung cancer, nasal cancer, breast cancer, cancer of the esophagus, and bladder cancer. In specific embodiments of the invention, the dye is IRdye800CW, IRdye800RS, IRdye700DX and/or a cyanine dye. In an embodiment of the invention, the retinoid analog has the formula:

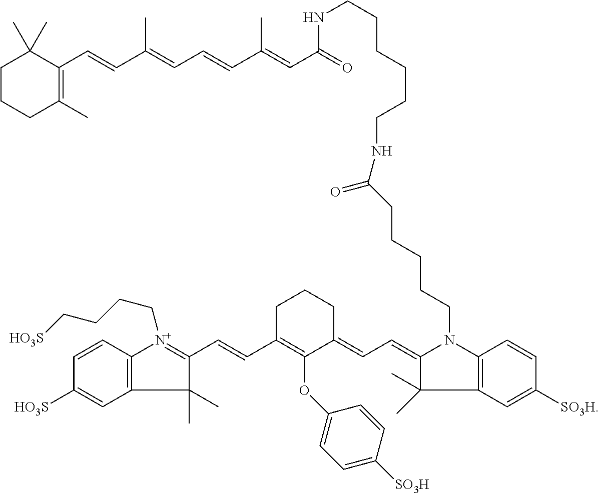

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows a confocal images of retinoids binding to human cancer cells and the retinoid imaging agent structure;

FIG. 2 shows the in vivo imaging of a human osteosarcoma tumor xenograft and side-by-side comparison of the active retinoid agent and free dye;

FIG. 3 shows the statistical comparison of the two groups of mice injected with either IRDye800CW or RA agent (compound 3) before or after 24 hours, as well as the vasculature agent signal intensity for all mice during the 3-day imaging study;

FIG. 4 shows the imaging results for human colon, lung, and nasal cancer, and neuroblastoma xenografts;

FIG. 5 shows the tumor-to-muscle ratio over time after a single injection of retinoid agent (animals shown in dorsal view);

FIG. 6 shows the plot of tumor-to-muscle (triangle) and liver-to-tumor ratio (square) versus time following injection;

FIG. 7 shows the tumor target response and systemic toxicity; FIG. 7A shows the loss of the dose response when increasing doses of the target-specific agent are used;

FIG. 7B shows the effect of increasing doses of retinoid agent on the gain of bodyweight as a measure of systemic toxicity;

FIG. 8 shows the in vivo whole body images (ventral view) and pathological results for the tumor (FIG. 8B), muscle (FIG. 8C), kidney (FIG. 8D), and liver (FIG. 8E).

### DETAILED DESCRIPTION OF THE INVENTION

It will be readily apparent to one skilled in the art that various embodiments and modifications may be made in the invention disclosed herein without departing from the scope and spirit of the invention.

### I. DEFINITIONS

In any embodiment herein, any R group (R₁, R₂, R₃, R₄, and/or R₅) may be further defined as alkyl_{(C=1-8)}, such as methyl, ethyl, n-propyl, or isopropyl. Any R group may comprise an alkenyl_{(C=1-8)} group, such as allyl. Any R group may comprise a substituted or unsubstituted aralkyl_{(C=1-8)} group, such as benzyl or 2-furanylmethyl.

In any embodiment herein regarding alkyl_{(C=1-8)}, aryl_{(C=1-8)} and aralkyl_{(C=1-8)} groups (e.g., alkyl_{(C=1-8)}, alkyl_{(C=1-8)} sulfonate, alkyl_{(C=1-8)} halide, aryl_{(C=1-8)} sulfonate, aralkyl_{(C=1-8)}, etc.), it is specifically contemplated that the number of carbons may be 1, 2, 3, 4, 5, 6, 7, or 8, or any range derivable therein. It is also specifically contemplated that any particular number of carbon atoms may be excluded from any of these definitions.

As used herein, “halide” means independently —F, —Cl, —Br or —I and “sulfonyl” means —SO₂—.

The term “alkyl,” when used without the “substituted” modifier, refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃. In certain embodiments, “lower alkyl” groups are contemplated, wherein the total number of carbon atoms in the lower alkyl group is 6 or less.

The term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “aryl,” when used without the “substituted” modifier, refers to a monovalent group, having an aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄—CH₂CH₃ (ethylphenyl), —C₆H₄—CH₂CH₂CH₃ (propylphenyl), —C₆H₄—CH(CH₃)₂, —C₆H₄—CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group, having an aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “aralkyl” when used without the “substituted” modifier refers to the monovalent group -alkanediyl-aryl, in which the terms alkanediyl and aryl are each used in a manner consistent with the definitions provided herein. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are (3-chlorophenyl)-methyl, 2-oxo-2-phenyl-ethyl (phenylcarbonylmethyl), 2-chloro-2-phenyl-ethyl and 2-methylfuranyl.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCH(F)— and —C≡CCH(Cl)— are non-limiting examples of substituted alkynediyl groups.

The terms “alkyl sulfonate” and “aryl sulfonate” refer to compounds having the structure —OSO₂R, wherein R is alkyl or aryl, as defined above, including substituted versions thereof. Non-limiting examples of alkyl sulfonates and aryl sulfonates include mesylate, triflate, tosylate and besylate. In certain embodiments, mesylates are excluded from compounds of the present invention.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxylic acid, ester, carbonyl, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups, including examples of their installation and removal, may be found in Greene and Wuts (1999), incorporated herein by reference in its entirety. The starting materials, products and intermediates described herein are also contemplated as protected by one or more protecting groups—that is, the present invention contemplates such compounds in their “protected form,” wherein at least one functional group is protected by a protecting group.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. Thus, in certain aspects, compounds of the present invention may comprise S- or R-configurations at particular carbon centers.

Persons of ordinary skill in the art will be familiar with methods of purifying compounds of the present invention. One of ordinary skill in the art will understand that compounds of the present invention can generally be purified at any step, including the purification of intermediates as well as purification of the final products. Purification procedures include, for example, silica gel column chromatography, HPLC, or crystallization. In particular embodiments, trituration is employed. In certain embodiments, solvent extraction is employed.

Modifications or derivatives of the compounds disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically-modified compound that still retains the desired effects or properties of the compound prior to the chemical modification. Using a retinoid imaging agent as an example, a “retinoid derivative” refers to a chemically modified retinoid imaging agent that still retains the desired effects of the parent retinoid imaging agent prior to its chemical modification. Such effects may be enhanced (e.g., slightly more effective, twice as effective, etc.) or diminished (e.g., slightly less effective, 2-fold less effective, etc.) relative to the parent retinoid imaging agent, but may still be considered a retinoid derivative. Such derivatives may have the addition, removal, or substitution of one or more chemical moieties on the parent molecule. Non-limiting examples of the types of modifications that can be made to the compounds and structures disclosed herein include the addition or removal of lower unsubstituted alkyls such as methyl, ethyl, propyl, or substituted lower alkyls such as hydroxymethyl or aminomethyl groups; carboxyl groups and carbonyl groups; hydroxyls; nitro, amino, amide, imide, and azo groups; sulfate, sulfonate, sulfono, sulfhydryl, sulfenyl, sulfonyl, sulfoxido, sulfonamide, phosphate, phosphono, phosphoryl groups, and halide substituents. Additional modifications can include an addition or a deletion of one or more atoms of the atomic framework, for example, substitution of an ethyl by a propyl, or substitution of a phenyl by a larger or smaller aromatic group. Alternatively, in a cyclic or bicyclic structure, heteroatoms such as N, S, or O can be substituted into the structure instead of a carbon atom.

Salts of any of the compounds of the present invention are also contemplated. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts, such as alkylammonium salts. Salts include, but are not limited to, sodium, lithium, potassium, amines, tartrates, citrates, hydrohalides and phosphates.

The term “DCM” as used herein refers to dichloromethane. The term “DIPEA” as used herein refers to N,N-Diisopropylethylamine, or Hünig's base. The term “DMAP” as used herein refers to 4-dimethylaminopyridine. The term “TFA” as used herein refers to trifluoroacetic acid. The term “DMF” as used herein refers to dimethylformamide.

Hydrates of compounds of the present invention are also contemplated. The term “hydrate” when used as a modifier to a compound means that the compound has less than one (e.g., hemihydrate), one (e.g., monohydrate), or more than one (e.g., dihydrate) water molecules associated with each compound, such as in solid forms of the compound.

It is contemplated that any embodiment discussed in this specification can be implemented with respect to any method, compound, or composition of the invention, and vice versa. Furthermore, compounds and compositions of the invention can be used to achieve methods of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### II. DISCUSSION OF GENERAL EMBODIMENTS

Like any new technology, the current molecular imaging must be combined with structural imaging data to determine the location and dimensions of the tumor. Cellular and histological data must be obtained and interpreted based on criteria developed over a hundred years ago, including cellular size and shape, nuclear morphology, cellular organization and whether there is invasion into the surrounding tissues.

The present disclosure provides a near-infrared (NIR)-labeled retinoid agents useful for in vitro and in vivo imaging studies. Furthermore, the imaging results were validated using pathological data. These results demonstrate that retinoid agents can bind to multiple cancer cell lines and detect multiple human cancer xenografts. The redistribution of the agent can be visualized within the body and the optimal biological dose that reduces the systemic toxicity can be determined. Also, these results demonstrate the feasibility of using image-guided therapy for personalized medicine.

Additionally, the present disclosure provides a NIR-labeled retinoid for use in in vitro and in vivo preclinical research. Using the optical properties of this agent, the trafficking of retinoids at the cellular level was studied. The NIR-labeled retinoid was used to detect multiple human cancer xenografts, and as a result, the unbound agent was associated with an increase in systemic toxicity. The amount of agent bound to the tumor is dependent on the agent's redistribution from the liver to the tumor over time. It was demonstrated that the target component of the agent is important to achieve a high tumor-to-background ratio (TBR). The therapeutic agents labeled with reporters provides information for determining the optimal dosing and scheduling which is beneficial for patients in this era of personalized and molecular medicine.

In general, the retinoid is coupled to a reporter. In some examples, the reporter is a dye that is compatible with NIR imaging. In specific examples, the dye is IRdye800CW, IRdye800RS, or IRdye700DX. Although in specific examples disclosed herein IRDye800CW is the reporter, one of ordinary skill in the art would readily recognize that this is a non-limiting example and other dyes may be used. For example, the chemical protocol disclosed herein is generalized and flexible enough to enable the skilled artisan to follow the protocol when it is desirous for labeling with other reporters. However, it must be noted that the binding affinity is altered in each cell line when the reporter was changed. The in vitro cell binding studies show that this agent strongly binds to all the tested human cancer cell lines except CM319 osteosarcoma cells. Confocal microscopy images reveal that the retinoid signal localizes to the cell membrane and cytoplasm. This binding and internalization are temperature-dependent; a higher signal intensity was observed at 37° C. than at 4° C. This temperature dependence suggests that the mechanism could involve receptor-ligand binding. The preferred method to demonstrate receptor specificity is to perform a blocking study using unlabeled retinoid analog and show that the labeled-retinoid analog binds to the same site as unlabeled retinoid analog. However, the low solubility of unlabeled retinoid analog made it impossible to carry out this study under consistent cell culture conditions. In order to perform the receptor specificity study, the structure of the retinoid analog must be modified to improve its solubility.

The side-by-side in vivo imaging study comparing free dye with the retinoid analog rule out the possibility of a free dye enhanced permeability and retention (EPR) effect on the imaging. The free dye was quickly cleared from the body within 24 hours following the injection because of the lack of a target component. The rapid signal intensity in both the liver and tumor demonstrates a pharmacodynamic difference between the free dye and retinoid analog. Both representative imaging results and population statistical analysis confirmed that the target component plays an important role in increasing the tumor-to-background ratio.

Multiple different tumor type positive imaging results indicates that this agent is useful as a non-disease specific imaging agent. For example, retinoid receptor-positive diseases are detectable with this imagining agent, such as, cancer, degenerative diseases, inflammation, cardiovascular and autoimmune diseases. Obviously this agent is not suitable for imaging liver disease because of the extensive signal intensity in this organ. The noninvasive in vivo studies demonstrate the importance of dynamic imaging and monitor the agent redistribution. Most of the agent initially remained in the liver, resulting in a very low tumor-to-muscle ratio. It took six days for this agent to gradually redistribute from the liver to the tumor, some animals reaching a maximum tumor-to-muscle ratio of 2.7. These data indicate that time is an important factor for both imaging and therapy. While it is common in current clinical practice to image all patients at the same time points, in is important to understand that this fixed time schedule could be misleading. As a result of the imaging studies, it was demonstrated that there are individual differences in optimal imaging times even amongst animals with the same genetic background. This indicates that there may be great variability among patients and that the possibility of positive results when imaging data are negative at one time point should not be excluded. It is desirable to perform imaging studies in orthotopic models. However, those models require long signal penetration depth for optical imaging and long half-life isotopes for nuclear medicine in order to image up to several days. Therefore, the results disclosed herein demonstrate the feasibility of imaging tumor xenografts using a reporter-labeled retinoid analog. In some examples, it may be desirous to apply this technique to superficial diseases, including but not limited to degenerative diseases, inflammation, cardiovascular and autoimmune diseases. In additional and alternative examples, it may be desirous to combine this technique with endoscopy in a clinical setting using optical modality.

Another significant finding from this study is that the signal intensity in the liver region had not returned to the muscle level by day 14, even after a single 18.4 μg injection of the retinoid analog imaging agent. These data demonstrate that this agent, like many other chemotherapeutic agents, can stay in the liver for a prolonged period of time. The long-term persistence of these agents in the liver could contribute to liver dysfunction.

Determining whether a disease marker can be saturated during target-specific therapy is an important issue to define in order to determine the effective biological dose. This is a common phenomenon for in vitro receptor-ligand binding assays. Since it was anticipated that such a situation would arise in the animal study, the tumor-to-muscle ratio was used to test the tumor dose acceleration response in a separate study. It was found that there was no statistically significant difference in the tumor-to-muscle ratio when the dose of the imaging agent was increased from 0.03 to 0.125 mg. These results support the hypothesis that a higher injection dose will not increase the tumor-to-muscle ratio tumor-to-muscle ratio once the tumor binding sites have been saturated. On the other hand, the unbound agent in the circulatory system could increase the background level, resulting in more systemic as well as organ-specific toxicity.

Bodyweight change is a very sensitive indicator for evaluating the systemic toxicity of a treatment regimen. It usually arises prior to permanent organ damage and must be assessed in every toxicity study according to the Food and Drug Administration (FDA) of the United States. Bodyweight gain was used as an indicator of systemic toxicity to compare the tumor saturation dose (0.1 mg/kg/week) with a dose that was five times lower than the standard therapeutic dose (0.6 mg/kg/week). It was found that the group receiving the 0.6 mg/kg/week dose had a significantly lower bodyweight gain as compared to the group receiving the tumor saturation dose (p=0.0001) or the untreated control group (p=0.0003). These results support the hypothesis that a target-specific biological agent should only be used at the biological response dose based on the availability of target molecules. Systemic toxicity will occur when the target is absent and/or the agent is overdosed.

Retinoids show a beneficial effect in the treatment and prevention of tumors in highly select patient populations (Camerini et al.; Khuri et al.; Tanaka et al.); however, this efficacy has not been confirmed in large-scale clinical trials, and all studies to date have shown a high frequency of serious adverse events. It is suspected that the failure of large trials to demonstrate any efficacy of retinoid treatment may stem from: (1) the molecular mechanisms underlying the anti-cancer effects of retinoids are poorly understood (Hansen et al.; Mongan et al.; Tanaka et al); (2) retinoid receptor status was not evaluated in the patients that participated in the clinical trials; (3) retinoid receptor changes after therapy were not evaluated; and (4) as with other chemotherapeutic agents, systemic toxicity can stem from the inability to determine the biological dose and distribution of the agent during the clinical trial.

Khuri and coworkers (Khuri et al.) reported results from a phase III clinical trial using a low-dose retinoid to prevent second primary tumors in patients with stage I and II head and neck cancer. Patients in the treatment group were given 30 mg/day of the drug. Assuming that the average bodyweight of these patients was 65 kg, they received 3.2 mg/kg/week of retinoid. Compared to the dose used in the animal study disclosed herein (0.74 mg/kg/week), this dose was 4.3 times higher by bodyweight or 292 times higher by body surface. If the agent in that clinical trial is similar to that used in the animal study disclosed herein in terms of its distribution and dynamic properties, it is unsurprising that 49.7% of those patients would develop adverse effects (Khuri et al.). This data demonstrate that it is important to apply target-specific therapy based on the molecular analysis and administrate the optimal biological dose instead of the maximum tolerated dose. Therefore, a noninvasive technique to detect retinoid receptor-positive tumors and to track the activity of retinoids is critically needed. An agent with “seek, treat, and see” capability will help in the future development of personalized molecular medicine.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods and Materials

Cell Lines.

The human colon (HT-29), nasal (CCL-30) and lung (A549) cancer cell lines were purchased from the American Type Culture Collection (ATCC, Manassas, Va.). Human osteosarcoma (F5M2, OS-9901, SOSP-S607, F4, CM319) and nasopharyngeal carcinoma cell lines (CNE-2) were generated in-house. The cells were cultured in Dulbecco's Modified Eagle Medium supplemented with high glucose and F12 nutrient (DMEM/F12, Invitrogen, Carlsbad, Calif.) with 10% fetal bovine serum (Hyclone, Logan, Utah) in a humidified incubator maintained at 37° C. with 5% CO₂.

Tumor Xenografts:

Four- to six-week-old male athymic nude mice (18-22 g) were housed and fed sterilized pellet chow and provided sterilized water. Animals were maintained in a pathogen-free mouse colony. The facility is accredited by the American Association for Laboratory Animal Care, and all experiments were performed in accordance with the guidelines of the Institutional Animal Care and Use Committee. Tumor cells were harvested near confluence by incubating them with 0.05% trypsin-EDTA. Cells were pelleted by centrifugation at 130×g for five minutes and resuspended in sterile phosphate-buffered saline (PBS). Tumors were induced in animals by subcutaneous injection of approximately one million tumor cells into the hind leg region of the mice. A total of 75 mice were used in this study.

Generating the NIR-Labeled Retinoid Agent for Imaging:

The target optical retinoid imaging agent was synthesized in three to four synthetic steps. In general, retinoic acid was reacted with N-hydroxysuccinimide in the presence of 1,3-diisopropylcarbodiimide and dimethylaminopyridine. The generated retinoic active (RA) ester was converted to RA-NH(CH2)6-NH-Trt by treatment with tritylhexane-1,6-diamine and N,N-diisopropylethylamine. The Trt-protecting group in this compound was removed under acidic conditions. The resulting compound was labeled with a NIR dye (IRDye800CW, IRdye800RS, or IRdye700DX) to yield the final NIR-labeled retinoid product.

Confocal Microscopy Imaging:

Cells were harvested, and the NIR-labeled retinoid agent or the free dye (100 μm) was added. The mixture was incubated for 60 minutes at 37° C. Cells were washed in PBS. Sytox Green in 95% ethanol was added, and cells were incubated for 15 minutes at 4° C. to fix them and stain their nuclei. Stained cells were then transferred to a slide and mounted for microscopic examination. Images were recorded by an Olympus confocal microscope. The microscope was equipped with an excitation (Ex) light source and emission (Em) filters to detect and separate the labeled retinoids (ex/em 785/810 nm) and cell nuclei (Ex/Em 488/510 nm) signals. Confocal image signals were recorded from one slice of a cell z-stack. Sytox green and NIR-labeled retinoid or NIR dye signals were pseudo-colored green (emission at 510 nm) and red (emission at 810 nm), respectively.

Animal Imaging:

The NIR-labeled retinoid agent was intraveneously (i.v.) injected into the tumor-bearing mice at a dose of 10 nmol for imaging. The animals were imaged from 1 to 9 days after the injection using an In-Vivo Multispectral System FX, a tandem (SPECT/CT) single photon emission computed tomography (SPECT) and computed tomography (CT) system and imaged with a positron emission tomography (PET) system.

Dose Acceleration Response and Toxicity:

Tumor-bearing mice were divided into three groups for the dose acceleration study; they were injected with 0.03 mg, 0.06 mg, or 0.125 mg of the NIR-labeled retinoid agent that was equivalent to the non-labeled retinoid, respectively. The tumor-to-muscle ratio was calculated from the imaging data. The systemic toxicity was measured using control mice. The mice were treated with PBS (control), a low dose (0.1 mg/kg/week), or a dosage that was five times lower than that reported in published human clinical trials (0.6 mg/kg/week). The bodyweight was measured every other day, and the percentage gain in bodyweight was used as the toxicity indicator.

Pathological Analysis:

Tissues were fixed with 10% Formalin. Paraffin embedded tissue blocks were cut and stained with haematoxylin and eosin (H&E).

Statistical Analysis:

Regions of interest were quantified from the in vitro and in vivo data using ImageJ (National Institutes of Health, USA). Data were analyzed using one-way (analysis of variance) ANOVA or general linear model (GLM). Data comparisons were illustrated in notched box-and-whisker plots. These plots provide the minimum, 25th percentile, median (central line), mean (+), 75th percentile and maximum. The medians (central lines) of the two box-and whisker plots were considered to significantly differ at the 0.05 level (95% confidence) if the corresponding notches did not overlap.

### Example 2

### Synthesis of Retinoic Acid Derivative

The reaction scheme and structure of retinoic acid derivative is shown in Scheme 1. Retinoic acid (compound 1) was reacted with 1-ethyl-3-(3′ dimethyl-aminopropyl)-carbodimide (EDC), N-hydroxysuccinimide (HOSu) and mono-tirtyl-1,6-diaminohexane acetic acid salt (H₂N(CH₂)₆NH-Trt) under basic conditions, followed by acidic deprotection to form compound 2. This compound was conjugated to IRDye800CW to result in the NIR optical imaging retinoic acid derivative (compound 3).

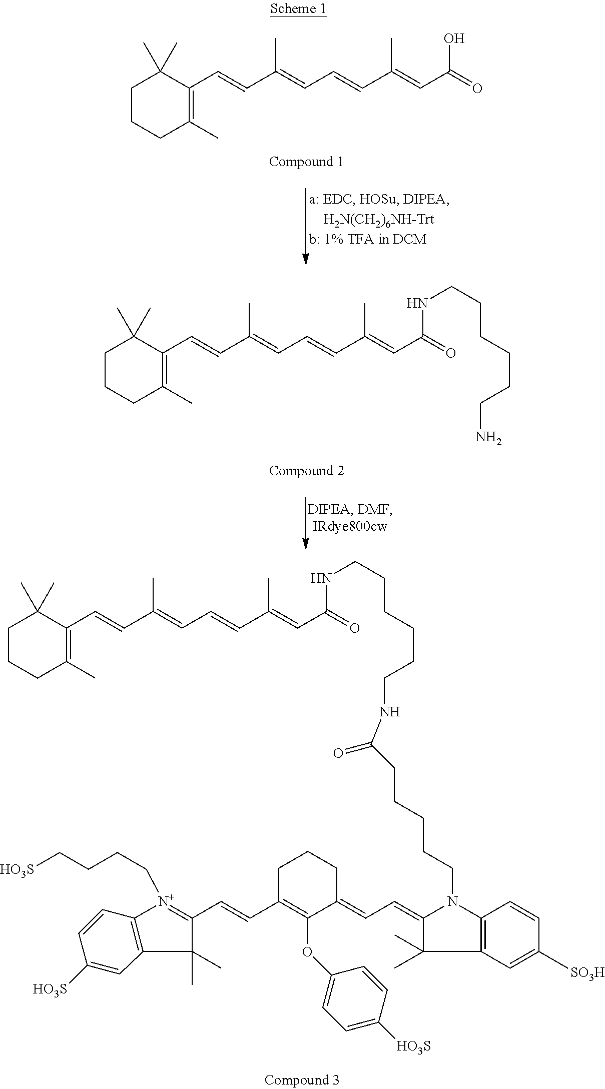

### Synthesis of N-(aminohexyl)retinoicamide (RA-NH(CH₂)₆NH₂)

Retinoic acid (120 mg, 0.4 mmol), EDC (120 mg, 0.6 mmol), HOSu (50 mg, 0.43 mmol), DIPEA (200 μL, 1.12 mmol) and DMAP (20 mg, 0.16 mmol) were dissolved in 5 mL of DCM After 3 hr stirring H₂N(CH₂)₆NH-Trt (150 mg, 0.4 mmol) was added to the solution. The mixture was stirred overnight at room temperature. The solvents were evaporated under vacuum, and the residue in ethyl acetate was washed with 5% NaHCO₃, 2% KHSO₄ and brine, and dried over MgSO₄. After removal of solvent the residue was stirred in 10 mL of 1% TFA in DCM for 15 minutes. The solvents were removed under vacuum. The product was purified by flash chromatography using ethyl acetate/methanol as eluent to yield compound 2.

### Synthesis of RA-HN(CH₂)₆NH—IRdye 800 (Compound 3)

Conjugation of IRdye 800CW (5 mg, 0.004 mmol) to the amino group of compound 2 or RA-NH(CH₂)₆NH₂ (4.4 mg, 0.01 mmol) was carried out in 1 mL of DIPEA/DMF(0.4 mL/9.6 mL) solution at 4° C. for two hours. The solvent was removed under vacuum and the residue was washed with ether several times. The solid was purified by reverse phase high pressure liquid chromatography (HPLC) and dried by lyophilization to yield the imaging agent (RA or compound 3).

### Example 3

### Retinoid Imaging Agent, In Vitro Cell Binding, and Effect of Temperature

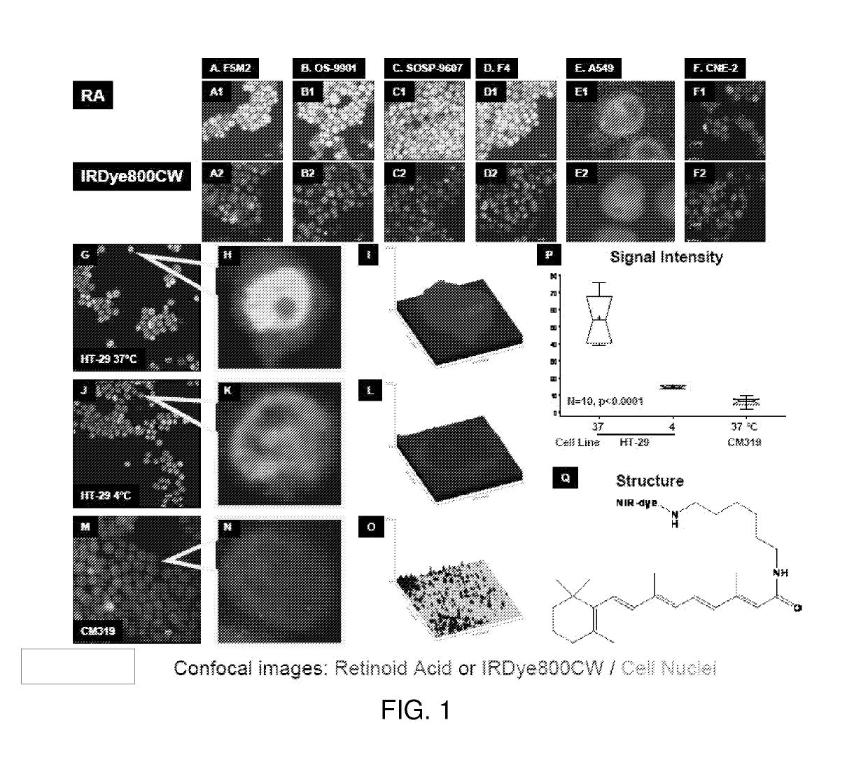

The uptake of NIR-labeled retinoid agent by human cancer cells was verified by confocal microscopy (FIG. 1A to 1H). The cell population image shows that this agent bound to human osteosarcoma (F5M2, OS-9901, SOSP-9607, and F4), human lung cancer (A549), human nasopharyngeal cancer (CNE-2), and human colon cancer (HT-29) cells (FIG. 1A1 to FIG. 1F1, FIG. 1G and FIG. 1H, red). In contrast, there was no detectable signal when these cell lines were incubated with free dye under the same conditions (FIG. 1A2 to FIG. 1F2). The retinoid signals were not only detected in the cytoplasm but also in the nuclei. The high magnification of a single cell confocal image shows that the retinoid signals localized to the cytoplasm (FIG. 1H, red). The nuclear internalization was temperature-dependent (FIG. 1J and FIG. 1K). The signal intensity was stronger in cells incubated at 37° C. (FIG. 1I) than in those incubated at 4° C. (FIG. 1L) or the negative cells incubated at 37° C. (FIG. 1M, FIG. 1N, and FIG. 1O). These differences are statistically significant (FIG. 1P). The structure of the retinoid imaging agent is provided in FIG. 1Q.

The cell population image shows that NIR-labeled retinoid agent binds to most cells (FIG. 1A1 to FIG. 1F1—top row and FIG. 1G, red signal) but not to IRDye800CW (FIG. 1A2 to FIG. 1F2—bottom row). FIG. 1E1 and FIG. 1H-red signal show the high magnification of a single cell showing the retinoid agent in the membrane and cytosolic compartments at 37° C., and FIG. 1J and FIG. 1K show that there is minimum binding when incubated at 4° C. The negative cells also show the minimum signal intensity (FIG. 1M and FIG. 1N). FIG. 1I, FIG. 1L, and FIG. 1O show the quantitative side-by-side comparison of the agent signal intensity on the same scale, and FIG. 1P shows that there is a statistically significant difference.

### Example 4

### In Vivo Imaging

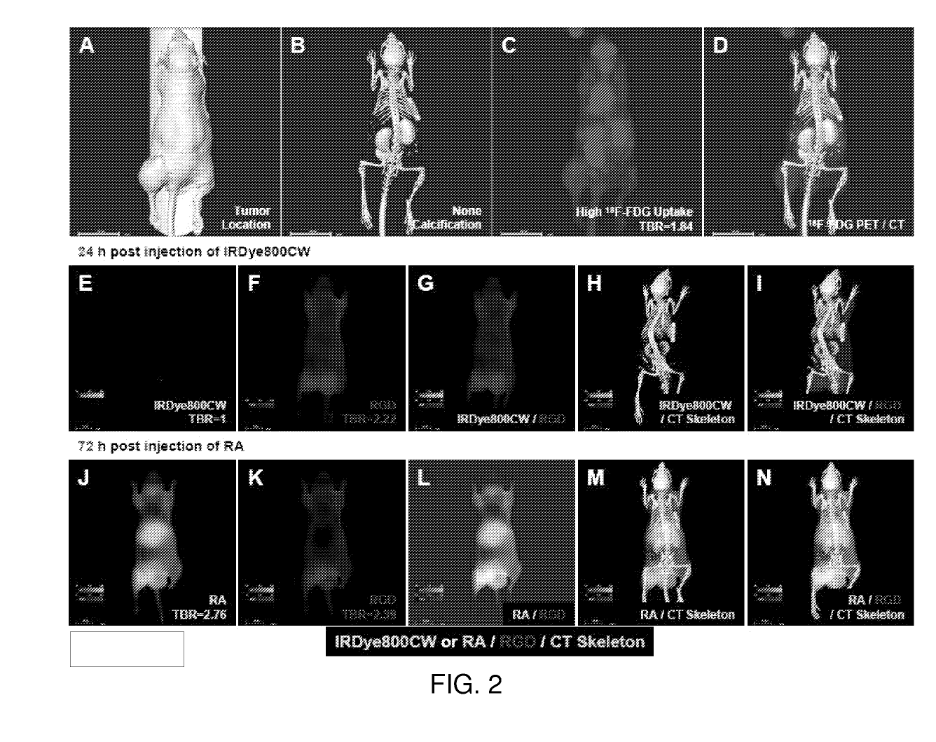

In general FIG. 2 shows the in vivo imaging of a human osteosarcoma tumor xenograft and side-by-side comparison of RA agent (compound 3) and free dye. The images show the tumor location and characteristics (FIG. 2A to FIG. 2D). FIG. 2E and FIG. 2G to FIG. 2I, green signal, shows the rapid clearance of IRDye800CW (24 hours) from the body, and FIG. 2F, FIG. 2G and FIG. 2I, red signal, show the remaining tumor vasculature imaging agent signal. FIG. 2J, FIG. 2L to FIG. 2N, green signal, shows the high signal intensity of the RA agent (compound 3) in the liver and tumor 72 hours post-injection, and FIG. 2K, red tumor, shows the vasculature imaging signal. FIG. 2L and FIG. 2N, yellow, show that the merged image is the tumor RA and vasculature signal overlay.

The F4 osteosarcoma tumor xenograft characteristics are provided in FIG. 2A to FIG. 2D. The tumor mass was located in the left hind region of the mouse (FIG. 2A). There was no calcification of the tumor mass (FIG. 2B). The tumor exhibited high glucose uptake (FIG. 2C). The merged image shows the ¹⁸F-FDG and CT skeleton results (FIG. 2D). To eliminate the potential well-established enhanced permeability and retention (EPR) effect of the imaging results, a side-by-side imaging comparison was performed to study and to directly compare the free dye (FIG. 2E to FIG. 2I) and compound 3 (FIG. 2J to FIG. 2M). The same intensity scale was used in this analysis. The optical signal was completely undetectable in the animal injected with free dye after 24 hours (FIG. 2E), whereas the animal injected with compound 3 yielded a tumor-to-background ratio (TBR) of 2.76 at 72 hours post-injection (FIG. 2J). The tumor vasculature imaging agent RGD, which was used as an injection control, shows that both animals exhibited similar TBR results (FIG. 2F and FIG. 2K). The merged RGD image with free dye (FIG. 2G) and compound 3 (FIG. 2L) shows the relationship of the tumor vasculature with different agents. The merged whole-body CT skeleton and IRDye800CW or compound 3 images show that the free dye was completely eliminated from the body (FIG. 2H). In addition, they demonstrate that the retinoid signal comes from the liver and the tumor (FIG. 2M). Finally, the merged free dye, RGD and CT images show the only high vasculature signal in the vicinity of the tumor (FIG. 2I). In contrast, the merged compound 3, RGD and CT images show both vasculature and RA (compound 3) signal intensity increases in the tumor region (FIG. 2N, yellow). All of the data were further analyzed at the animal population level.

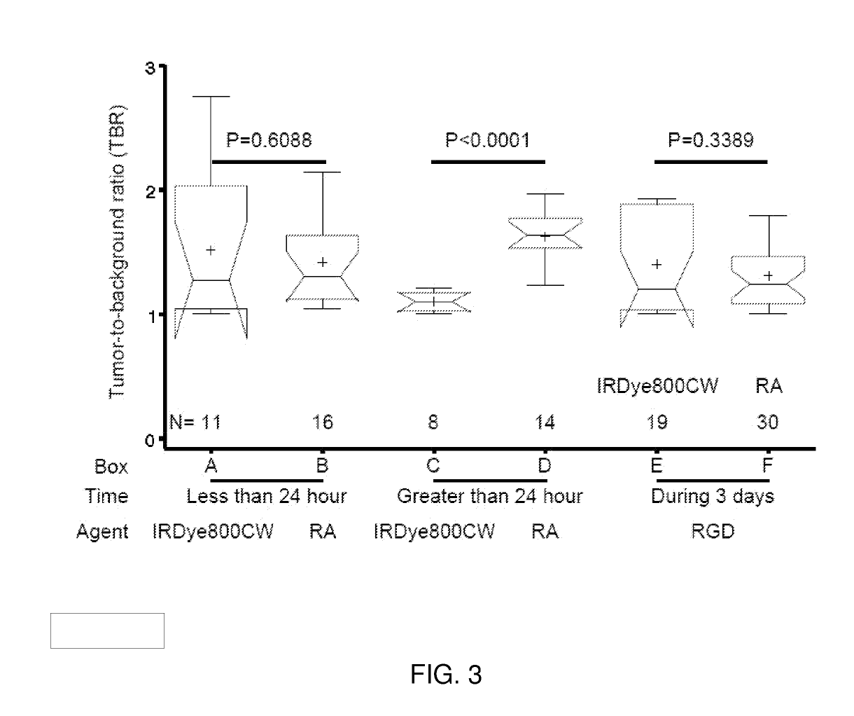

The tumor-to-background ratio (TBR) results are provided in FIG. 3. There were two groups of mice injected with either IRDye800CW (FIG. 3, box A) or RA agent (FIG. 3, box B). There was no statistically significant difference in the signal intensity within the first 24 hours (P=0.6088). The signal intensity of the IRDye800CW group (FIG. 3, box C) was significantly lower than the RA group (FIG. 3, box D) after 24 hours (P<0.0001). There was no statistically significant difference in the RGD signal intensity between the two groups (FIG. 3, box E and F respectively) during the duration of the 3-day imaging study (P=0.3389).

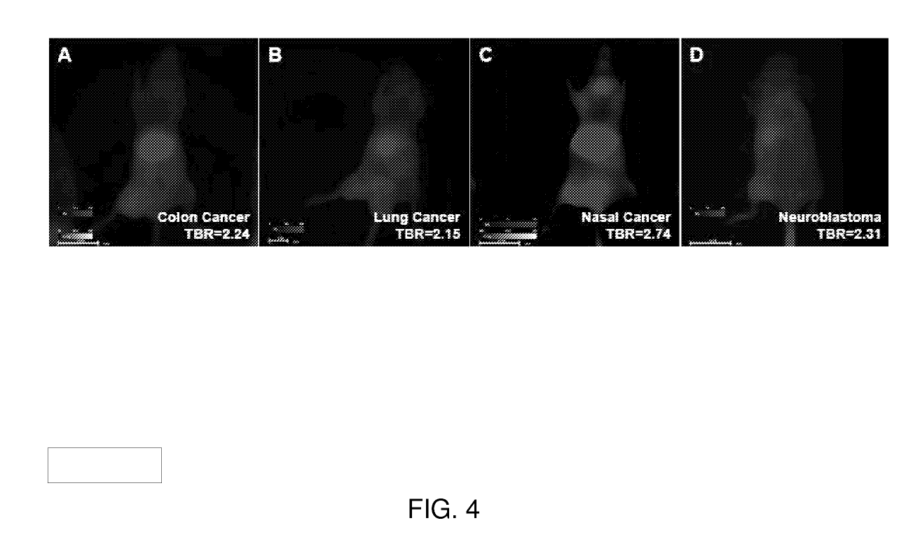

To confirm the findings in other cancer cell types, the RA agent was tested in human colon (FIG. 4A), lung (FIG. 4B), nasal (FIG. 4C) cancer, and neuroblastoma (FIG. 4 D) xenografts. The RA agent bound to all four tumor xenografts and yielded a tumor-to-background ratio ranging from 2.15 to 2.74.

### Example 5

### Tumor-to-Muscle and Liver-to-Tumor Ratios

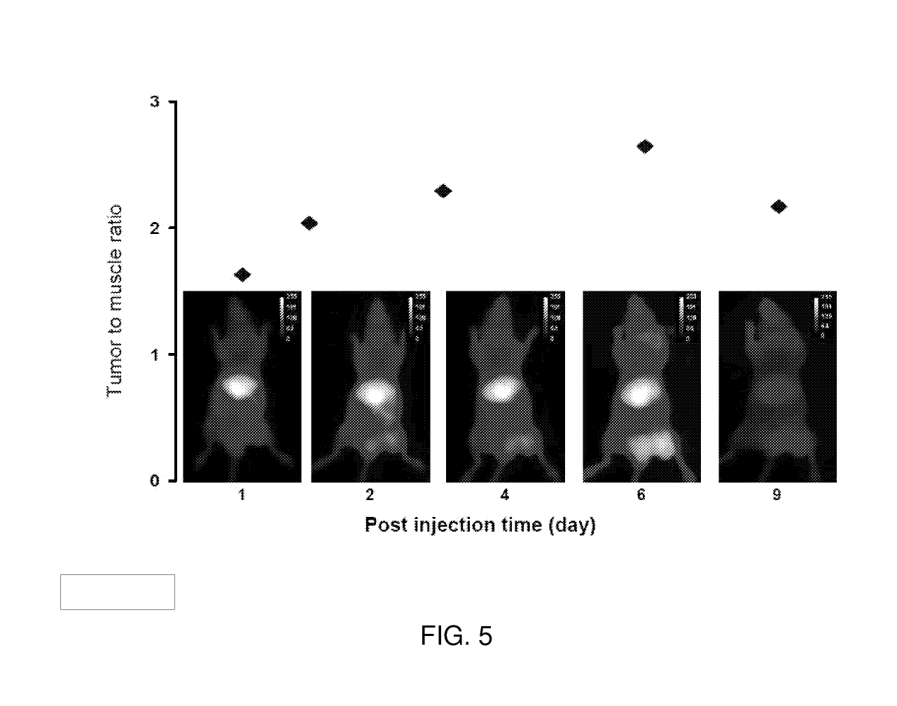

To determine the optimal imaging time, the imaging of the human colon cancer-bearing mice was done for up to 9 days and the tumor-to-muscle ratio was calculated. FIG. 5 shows a single mouse imaging result after a single injection of 18.4-μg of NIR-labeled retinoid agent per 25 grams of bodyweight. The plot shows the tumor-to-muscle ratio (TMR) and corresponding whole body NIR imaging results. The imaging agent was initially concentrated in the liver after the injection, but it gradually redistributed from the liver to the tumor over time. This redistribution resulted in a corresponding increase in the TMR. The maximum TMR (2.7) was reached 6 days post-injection in some animals, after which it started to decrease. However, the signal in the liver was still higher than the background level at 14 days post-injection (data not shown).

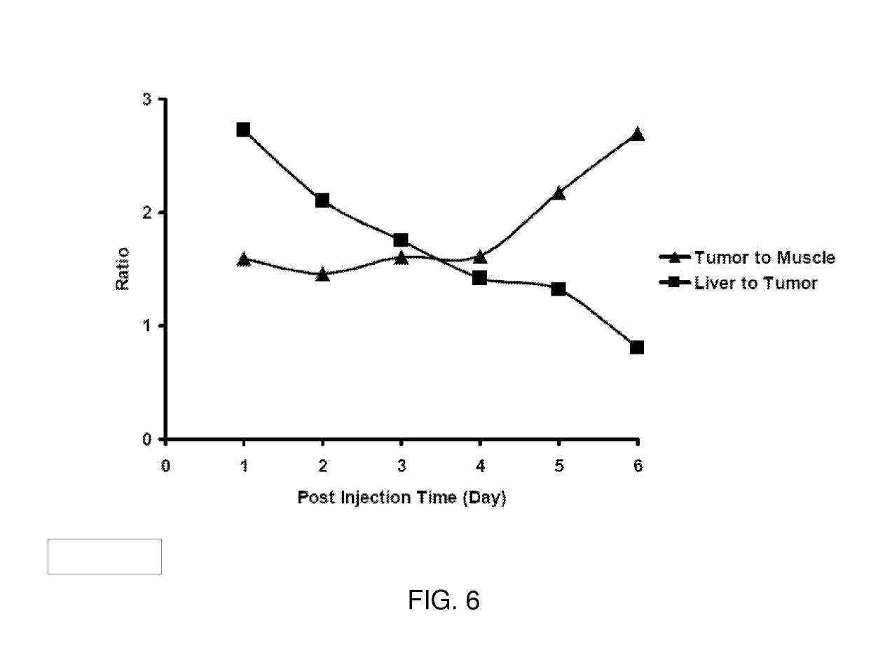

FIG. 6 illustrates this dynamic change by plotting the tumor-to-muscle ratio (in triangles) versus the liver-to-tumor ratio (in squares) over time. The liver-to-tumor ratio was almost twice as high as the tumor-to-muscle ratio 1 day post-injection. These ratios intersected 3 days post-injection. The tumor-to-muscle ratio then quickly increased and reached its maximum value 6 days post-injection.

### Example 6

### Dose Response and Systemic Toxicity

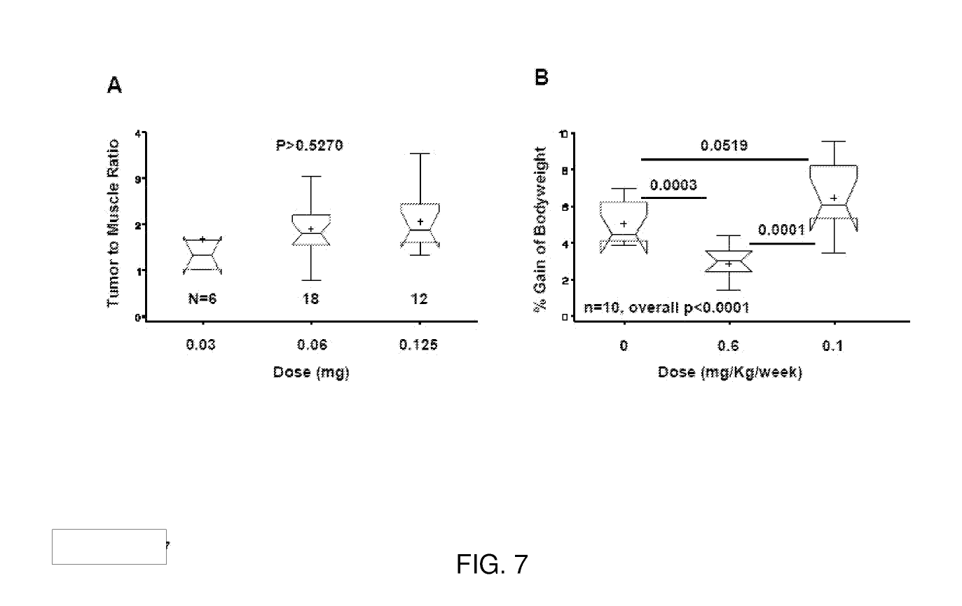

To test for a saturation effect of the target-specific biological agent, three groups of animal were injected with different doses of the retinoid agent. The tumor-to-muscle ratio was used to compare the dose response in the tumor. There was no significant difference when the injection dose was increased from 0.03 to 0.125 mg (p>0.5270) (FIG. 7A). However, the higher dose was associated with a significant decrease in the gain of bodyweight (FIG. 7B). The percentage of bodyweight gain in animals in the high dose (0.6 mg/kg/week) group was significantly lower than the gain observed in the untreated control group (p=0.0003) or in the low dose (0.1 mg/kg/week) treatment group (p=0.0001). There were no differences between the control and low dose treatment groups (p=0.0519).

### Example 7

### Pathology

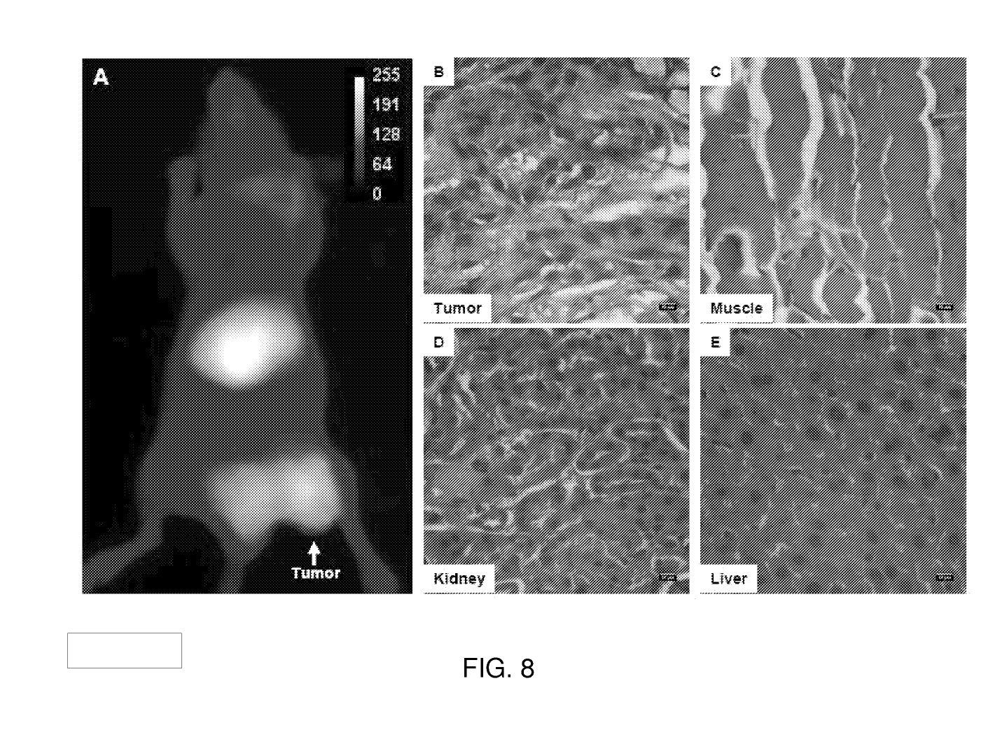

To verify the whole body imaging results (FIG. 8A) at a pathological level, H&E staining was performed on the tumor (FIG. 8B), muscle (FIG. 8C), kidney (FIG. 8D), and liver (FIG. 8E) tissues.

### Example 8

### Synthesis of NIR Labeled Retinoid Analogs

The imaging agent has the following general structure:

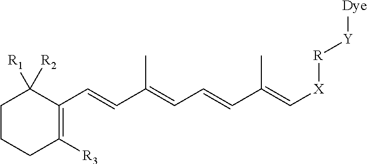

In the general formula for the imaging agent, the general linker moiety is represented by X—R—Y. In some examples, X is chemically bonded to retinoid analog through amide, amine, ester, ether, thioether, carbonyl, a carbon-carbon single bond, a carbon-carbon double bond, or a carbon-carbon triple bond. The spacer moiety is represented by R. In some instances, R is alkyl_{(C=1-8)}, alkenyl_{(C=1-8)}, or aralkyl_{(C=1-8)}, or a substituted version of any of these groups. The type of chemical bond used to connect the spacer moiety to the dye may be varied. This chemical bond is represented by Y. In some cases, the spacer moiety is bonded to the dye through an amide or a thioether bond. In specific examples, the dye is IRdye800CW, IRdye800RS, or IRdye700DX.

In general, R₁, R₂ and R₃ may be independently selected from alkyl, aryl, aralkyl, alkyl halide, alkenyl, substituted alkyl, substituted aryl, substituted aralkyl, substituted alkyl halide, substituted alkenyl or any combinations thereof. In some examples, R₁, R₂ and R₃ may be independently selected from alkyl_{(C=1-8)}, aryl_{(C=6-18)}, aralkyl_{(C=7-15)}, alkyl halide_{(C=1-8)}, alkenyl_{(C=1-8)}, substituted alkyl_{(C=1-8)}, substituted aryl_{(C=6-18)}, substituted aralkyl_{(C=6-18)}, substituted alkyl halide_{(C=1-8)}, substituted alkenyl_{(C=2-10)} or any combinations thereof. In specific examples, R₁, R₂ and R₃ are alkyl_{(C=1-8)}. In particular examples, R₁, R₂ and R₃ are methyl.

In general, the imaging agent can be synthesized in two major steps, as shown in Scheme 2. Although Scheme 2 only shows the two major steps, one of ordinary skill in the art would readily recognize that steps such as protections, deprotections and the arrangement of the synthetic process can be modified to arrive at the same imaging agent. For example, in some cases it may be synthetically advantageous to perform step 2 before step 1 and/or include and additional step 3 in order to facilitate purification. In some cases, it may be advantageous to use a protecting agent other than—Trt. There are a wide range variations that can be made to the synthetic route shown in Scheme 2 and still be within the scope of the present invention.

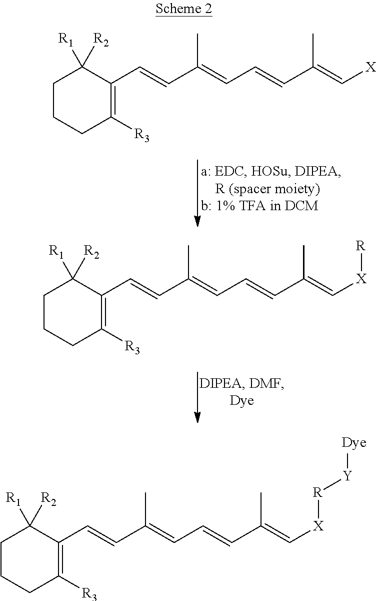

In general, Step 1 comprises coupling the retinoid analog and the linker moiety. The retinoid analog and the linker moiety may be coupled through an amide, an amine, an ester, an ether, thioether, carbon-carbon single bond, carbon-carbon double bond, or a carbon-carbon triple bond which is represented by X.

In the specific example shown below, the retinoid analog is coupled to the linker moiety through an amide bond.

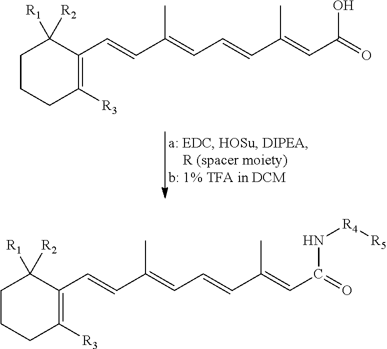

In this specific example, the coupling is accomplished through a two step process. The reaction conditions for the first step include adding EDC, HOSu, DIPEA to the linker moiety. In this example, the linker moiety, R, is a primary amine represented by the formula R₄-R₃—NH₂. The second step of the coupling process includes treating the reaction mixture with 1% TFA in DCM.

In some examples, R₄ is alkyl_{(C=1-8)}, alkenyl_{(C=1-8)}, or aralkyl_{(C=1-8)}, or a substituted version of any of these groups, and R₅ is —NH₂ or —SH. In the specific case where the retinoid analog and the linker moiety are connected by an amine, R is a primary amine represented by the formula R₄-R₃—NH₂, and the coupling step is followed by a reductive amination of the carbonyl. In the specific case where the retinoid analog and the linker moiety are connected by an ester, R is a primary alcohol represented by the formula R₄-R₃—OH. In the specific case where the retinoid analog and the linker moiety are connected by an ether linkage, R is a primary alcohol represented by the formula R₄-R₃—OH, and the coupling is followed by treating the ester with BF₃-etherate and either LiAlH₄, LiBH₄ or NaBH₄ with trichlorosilane and UV light and with catalytic hydrogenation. In the specific case where the retinoid analog and the linker moiety are connected by an thioether linkage, R is a primary thiol represented by the formula R₄-R₃—SH, and the coupling is followed by treating the thioester with BF₃-etherate and either LiAlH₄, LiBH₄ or NaBH₄ with trichlorosilane and UV light and with catalytic hydrogenation. In the specific case where the retinoid analog and the linker moiety are connected by an carbonyl (ketone) linkage, R is a lithium dialkylcopper reagent (R₂CuLi) represented by the formula (R₄-R₃)₂CuLi, and the coupling is preceded by converting the retinoid analog with thionyl chloride to form the retinoid analog acyl chloride.

In the specific case where the retinoid analog and the linker moiety are connected by a carbon-carbon double bond, R is a primary halide represented by the formula R₄-R₃—Cl, and the prior to coupling the primary halide is treated with triphenylphosphine, a base then added to the aldehyde of the retinoid analog which is followed by hydrolysis. The specific cases where the retinoid analog and the linker moiety are connected by a carbon-carbon single bond or carbon-carbon triple bond, only requires converting the carbon-carbon double bond of the above mentioned retinoid to either a single bond or a triple bond.

In particular examples, X is —NHCO—, —NH—, —OCO—, —O—, —S—, —CH₂CH₂—, —CH═CH—, or —C≡C—. In general, R is alkyl_{(C=1-8)}, alkenyl_{(C=1-8)}, or aralkyl_{(C=1-8)}, a substituted version of any of these groups, or a substituted and functionalized version of any of these groups. In some examples, R is further defined by the formula R₄-R₃—W where W is an amino (—NH₂), a halo (—Cl, —Br, —I), a hydroxy (—OH), thio (—SH), or some other functionality as required to form an amide, amine, ester, ether, thioether, carbonyl, a carbon-carbon single bond, a carbon-carbon double bond, or a carbon-carbon triple bond. In particular examples, R₃ is alkyl_{(C=1-8)}, alkenyl_{(C=1-8)}, or aralkyl_{(C=1-8)}, a substituted version of any of these groups, or a substituted and functionalized version of any of these groups. In specific examples, R₄ is —NH₂ or —SH. In specific examples, the dye is IRdye800CW, IRdye800RS, or IRdye700DX.

In a specific example, X is an amide, R is —(CH₂)₆—, Y is an amide, the dye is IRdye800CW and the composition has the formula:

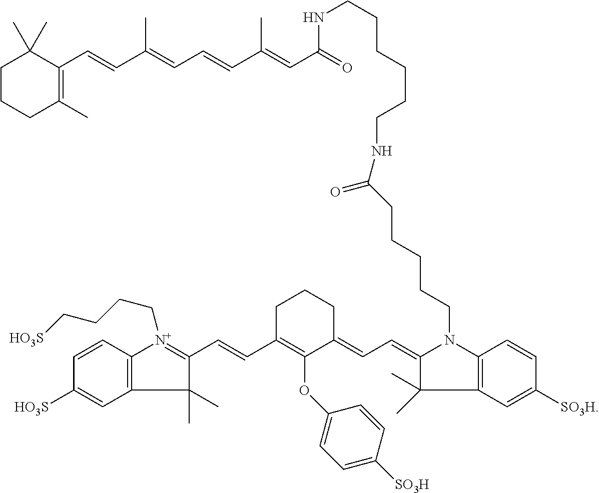

### IV. REFERENCES

The following references, to the extent that they provide exemplary procedural or other details supplementary to those set forth herein, are specifically incorporated herein by reference.
Hansen L A, Sigman C C, Andreola F, Ross S A, Kelloff G J, De Luca L M. Retinoids in chemoprevention and differentiation therapy. Carcinogenesis 2000; 21:1271-1279.
Mongan N P, Gudas L J. Diverse actions of retinoid receptors in cancer prevention and treatment. Differentiation 2007; 75:853-870.
Camerini T, Mariani L, De Palo G, et al. Safety of the synthetic retinoid fenretinide: long-term results from a controlled clinical trial for the prevention of contralateral breast cancer. J Clin Oncol 2001; 19:1664-1670.
Khuri F R, Lee J J, Lippman S M, et al. Randomized phase III trial of low-dose isotretinoin for prevention of second primary tumors in stage I and II head and neck cancer patients. J Natl Cancer Inst 2006; 98:441-450.
Tanaka T, Suh K S, Lo A M, De Luca L M. p21WAF1/CIP1 is a common transcriptional target of retinoid receptors: pleiotropic regulatory mechanism through retinoic acid receptor (RAR)/retinoid X receptor (RXR) heterodimer and RXR/RXR homodimer. J Biol Chem 2007; 282:29987-29997.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A composition comprising:
a retinoid analog
a spacer moiety comprising a reactive amino functionality wherein the spacer moiety is chemically bonded to the retinoid analog; and,
a dye that is chemically bonded to the spacer moiety.

2. The composition of claim 1 wherein the chemical bond connecting the retinoid analog and the spacer moiety forms a ester, ether, thioether, thioester, amide, a carbon-carbon single bond, a carbon-carbon double bond, or a carbon-carbon triple bond.

3. The composition of claim 2 wherein the spacer moiety further comprises an alkyl_{(C=1-8)}, alkenyl_{(C=1-8)}, or aralkyl_{(C=1-8)}, or a substituted version of any of these groups.

4. The composition of claim 2 wherein the dye is a contrast agent.

5. The compositions of claim 4 wherein the dye is IRdye800CW.

6. The composition of claim 4 wherein the dye is IRdye800RS.

7. The composition of claim 4 wherein the dye is IRdye700DX.

8. The composition of claim 1 wherein the retinoid analog has the formula:

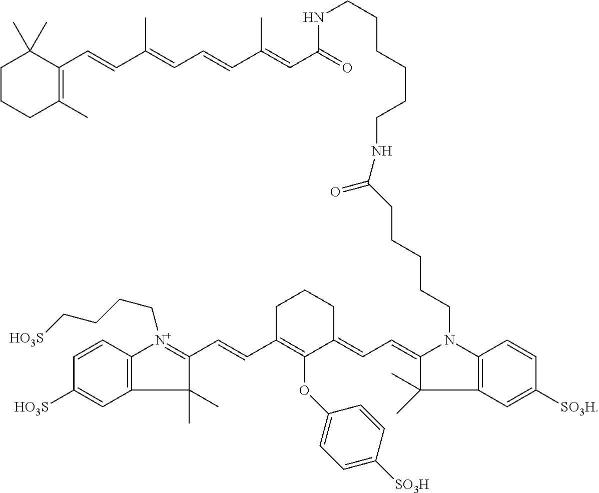

9. A composition having the general formula: 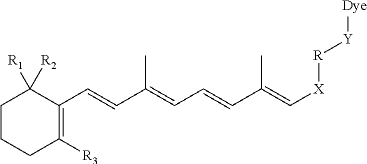
wherein
R₁, R₂ and R₃ may be independently selected from alkyl_{(C=1-8)}, aryl_{(C=6-18)}, aralkyl_{(C=7-15)}, alkyl halide_{(C=1-8)}, alkenyl_{(C=1-8)}, substituted alkyl_{(C=1-8)}, substituted aryl_{(C=6-18)}, substituted aralkyl_{(C=6-18)}, substituted alkyl halide_{(C=1-8)}, substituted alkenyl_{(C=2-10)} or any combinations thereof;
X is —NHCO—, —NH—, —OCO—, —O—, —S—, —CH₂CH₂—, —CH═CH—, or —
C≡C—;
R is alkyl_{(C=1-8)}, alkenyl_{(C=1-8)}, or aralkyl_{(C=1-8)}, a substituted version of any of these groups, or a substituted and functionalized version of any of these groups;
Y is —NHCO— or —SCO—; and,
dye is a fluorophore.

10. The composition of claim 9, wherein R is further defined by the formula R₅-R₄—W, wherein
W is —NH₂, a halogen, a hydroxy group, thiol and wherein W is coupled to X an amide, amine, ester, ether, thioether, ketone, a carbon-carbon single bond, a carbon-carbon double bond, or a carbon-carbon triple bond is formed;
R₄ is alkyl_{(C=1-8)}, alkenyl_{(C=1-8)}, or aralkyl_{(C=1-8)};
R₅ is —NH₂ or —SH and when coupled to the dye forms an amide bond or a thioester bond.

11. The composition of claim 10, wherein X and W forms an amide bond.

12. The composition of claim 11, wherein R₄ is alkyl_{(C=5-8)}.

13. The composition of claim 12, wherein Y is —NHCO—.

14. The composition of claim 13, wherein the dye is IRdye800CW.

15. The composition of claim 14 having the formula:

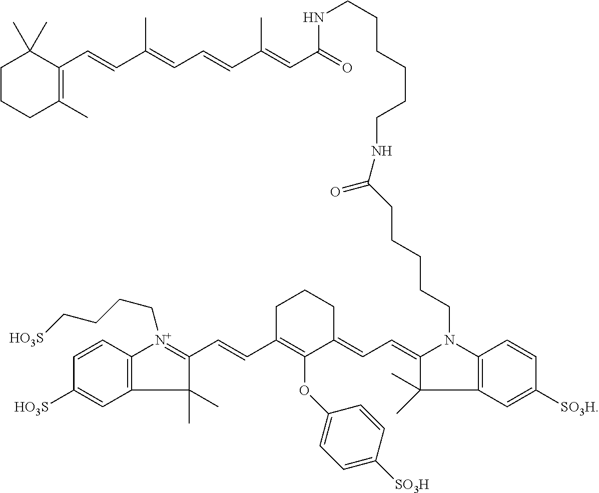

16. A method for near infrared imaging comprising the steps of:
treating a subject with a compound having the general formula: 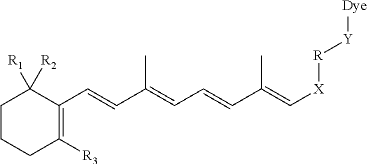
wherein
R₁, R₂ and R₃ may be independently selected from alkyl_{(C=1-8)}, aryl_{(C=6-18)}, aralkyl_{(C=7-15)}, alkyl halide_{(C=1-8)}, alkenyl_{(C=1-8)}, substituted alkyl_{(C=1-8)}, substituted aryl_{(C=6-18)}, substituted aralkyl_{(C=6-18)}, substituted alkyl halide_{(C=1-8)}, substituted alkenyl_{(C=2-10)} or any combinations thereof;
X is —NHCO—, —NH—, —OCO—, —O—, —S—, —CH₂CH₂—, —CH═CH—, or —C≡C—;
R is alkyl_{(C=1-8)}, alkenyl_{(C=1-8)}, or aralkyl_{(C=1-8)}, a substituted version of any of these groups, or a substituted and functionalized version of any of these groups;
Y is —NHCO— or —SCO—; and,
dye is a fluorophore.

17. The method of claim 16, wherein the dye is IRDye800CW.

18. The method of claim 16 wherein the compound has the formula:

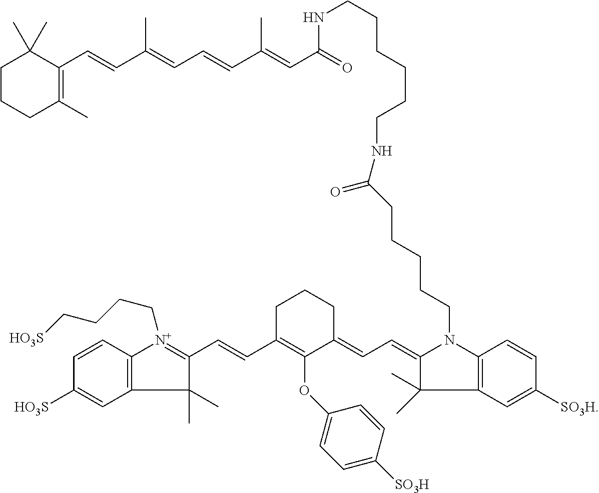

19. The method of claim 18, wherein the compound is non-radioactive.

20. The method of claim 16, wherein the dye is a cyanine dye.
